(19) United States
(12) Patent Application Publication
(10) Pub. No.: US 2026/0286398 A1
(43) Pub. Date: Sep. 24, 2026

(54) METHOD FOR TREATING A LIGNOCELLULOSIC BIOMASS

(71) Applicant: IFP ENERGIES NOUVELLES, Rueil-Malmaison (FR)

(72) Inventors: Matthieu ARAGONES, Rueil-Malmaison Cedex (FR); Meriem BOURAS, Rueil-Malmaison Cedex (FR); Gilles FERSCHNEIDER, Rueil-Malmaison Cedex (FR)

(73) Assignee: IFP ENERGIES NOUVELLES, Rueil-Malmaison (FR)

(21) Appl. No.: 19/476,333

(22) PCT Filed: May 2, 2024
(86) PCT No.: PCT/EP2024/062128

§ 371 Date: Oct. 17, 2025

(30) Foreign Application Priority Data
May 23, 2023 (FR) 2305093

(51) Int. Cl. C12P 7/08; C12M 1/00; C12M 1/40; C12P 19/02

(52) U.S. Cl. CPC C12P 7/08; C12M 21/18; C12M 45/20; C12P 19/02; C12P 2201/00

(57) ABSTRACT

A process for treating lignocellulosic biomass comprises:
b) acidic or neutral pretreatment of the biomass to produce an acidic or neutral pretreated biomass,
alternating with
b′) basic pretreatment of the biomass previously placed under acidic, neutral or basic conditions to produce a basic pretreated biomass,
followed by
c) enzymatic hydrolysis of the acidic pretreated biomass and/or of the basic pretreated biomass, to obtain a hydrolyzed biomass,
d) solid/liquid separation of the hydrolyzed biomass obtained at the end of step c) in the form of sugar(s) or a step of solid/liquid separation of the hydrolyzed biomass in the form of sugar(s) obtained at the end of step c) then treated in steps subsequent to the enzymatic hydrolysis step c), in order to obtain a separated juice and an unconverted solid residue, and
e) recycling the solid residue to basic pretreatment step b′).

### TECHNICAL FIELD

The invention relates to a process for treating lignocellulosic biomass to produce “second-generation” (2G) sugar juices. These sugar juices may be used to produce other products via a biochemical pathway, especially by fermentation (for example alcohols such as ethanol and butanol), or other molecules, for example solvents such as acetone, etc.

### PRIOR ART

Lignocellulosic biomass represents one of the most abundant renewable resources on Earth. The substrates considered are very varied, they relate both to ligneous substrates such as various woods (hardwoods and softwoods), coproducts obtained from agriculture (wheat straw, corn cobs, etc.) or from other industries: agrifood, paper, lignocellulosic waste, etc.

The process for treating lignocellulosic biomass generally comprises:
pretreatment of the biomass by cooking, optionally coupled with steam explosion, and generally preceded by impregnation of the biomass with an acidic, basic, neutral or oxidizing liquor,
enzymatic hydrolysis resulting in the production of sugar juices generally based on C5 and C6 sugars (i.e. sugars having 5 or 6 carbons),
and optionally fermentation of these sugars by a yeast to convert them into an alcohol of ethanol type.

The process also includes steps of separating and/or purifying the target end product (sugar, alcohol, solvent, etc.).

Lignocellulosic biomass is composed of three main polymers: cellulose (35% to 50% by weight), which is a polysaccharide essentially consisting of hexoses; hemicellulose (20% to 40% by weight), which is a polysaccharide essentially consisting of pentoses; and lignin (10% to 30% by weight), which is a polymer of complex structure and high molecular weight composed of aromatic alcohols connected via ether linkages. These different molecules are responsible for the intrinsic properties of the plant wall and are organized in a complex tangle.

Of the three base polymers that make up lignocellulosic biomass, it is cellulose and hemicellulose that make possible the production of 2G sugar juices.

Usually, hemicellulose is predominantly broken down into sugar during the pretreatment, and cellulose is converted into sugar (glucose) by enzymatic hydrolysis. However, crude cellulose remains difficult for enzymes to access, hence the need for a pretreatment. This pretreatment makes it possible to modify the physicochemical properties of the lignocellulosic biomass in order to improve the accessibility of the cellulose to the enzymes and its reactivity to enzymatic hydrolysis.

There are many technologies which are advantageous to the invention for performing this pretreatment, which will be combined below under the generic term “cooking”: acidic cooking, alkaline cooking, cooking by autohydrolysis, steam explosion, and “organosolv pulping” processes. The latter process involves a pretreatment in the presence of one or more organic solvents and generally water. The solvent can be an alcohol (ethanol), an acid of acetic acid or formic acid type, or also acetone. “Organosolv pulping” processes result in at least partial dissolution of the lignin and partial solubilization of the hemicelluloses. There are thus two outlet streams: the pretreated substrate comprising residual cellulose, hemicellulose and lignin, and the solvent phase which contains the solubilized lignin and part of the hemicelluloses. There is generally a step of regeneration of the solvent, which makes it possible to extract a lignin stream. Certain “organosolv pulping” treatments (especially with ethanol) are coupled with the addition of a strong acid (such as H₂SO₄). It is also possible to consider contacting the biomass with the solvent via an impregnation reactor before the cooking phase or contacting the biomass with the acid catalyst before carrying out “organosolv pulping” cooking.

Various configurations are reported for example in the document “Production of Bioethanol from Lignocellulosic Materials via the Biochemical Pathway: A Review”, M. Balat, Energy Conversion and Management, 52 (2011), 858-875, or also in the document “Bioethanol Production from Agricultural Wastes: An Overview”, N. Sarkar, S. Kumar Ghosh, S. Bannerjee and K. Aikat, Renewable Energy, 37 (2012), 19-27.

One of the most effective pretreatments is steam explosion, in particular under acidic conditions, which makes possible virtually complete hydrolysis of hemicellulose and a significant improvement in the accessibility and the reactivity of cellulose to enzymes. This pretreatment can be preceded by other treatment(s).

When a treatment requires a pressure step (impregnation, cooking-type pretreatment or the like), it is necessary to make use of means of introducing solid biomass that are compatible with these pressure steps. This is the case, for example, with compression screws, an embodiment of which is described in U.S. Pat. No. 4,599,138.

Patent FR 3 075 203 describes a process comprising impregnation of the biomass with an acidic liquor, followed by cooking and steam explosion of the impregnated biomass, with an adjustment of the acidity of the acidic liquor and recycling thereof. Patent FR 3 075 201 likewise describes a process of pretreatment of the biomass by acid impregnation, followed by steam explosion, with additional washing of the reactor feed means and recycling of the wash waters to the process.

These various types of process starting from lignocellulosic biomass generate solid lignin-based residues, in particular after enzymatic hydrolysis and/or after a fermentation when this is followed by conversion of the sugars into alcohol in particular.

These solid residues, which will be referred to in the present text as “unconverted solid residue” or “lignin cake”, are rich in lignin, which shows little or no reactivity to the action of the enzymes used for hydrolysis of the biomass, which are generally cellulases and hemicellulases. These wood residues can be used in particular as fuel or can be incorporated as a filler in resin-based or bitumen-based products, for example.

However, these unconverted residues do not consist (aside from a certain amount of water) solely of lignin: they may also contain hemicellulose and/or cellulose that have not reacted in the hydrolysis. And removing these residues from the biomass conversion process means losing these fractions of unreacted polymeric sugars, which impacts the biomass to sugar (or alcohol) conversion efficiency.

It has already been proposed to reuse these types of solid residues in the biomass treatment process: patent EP 2 516 661 proposes a biomass treatment process with a pretreatment of the biomass in an alkaline medium, enzymatic hydrolysis, fermentation of the hydrolysate obtained to obtain an alcohol-containing fermentation must, separation/purification of the alcohol, and separation of a residue cake. This residue cake is sent to a separate cellulose regeneration reactor where the cake is mixed with an alkaline solution and heated before being recycled downstream of the pretreatment. This solution is advantageous, since it opts to recover the remaining cellulose contained in this woody cake in order to reincorporate it into the process and so increase the overall biomass conversion efficiency. However, it necessitates a separate treatment of this cake using an additional reactor that is dedicated thereto and that will carry out an alkaline cooking of the cake before recycling it, which increases the plant costs and operating costs of the process as a whole and increases the complexity of its implementation.

Another solution has been proposed in patent EP 2 430 171, which is quite similar to the preceding one but providing an acidic rather than alkaline cooking of the wood residue, with the same drawbacks.

These solutions for reusing these residues are advantageous, but remain complex to implement.

Furthermore, reactors for pretreatment by cooking, particularly with steam explosion, experience problems of gradual fouling, with deposits of solid residues on their walls that accumulate and can progressively give rise to operational problems (clogging of the reactor inlets/outlets, greater difficulty in circulating the biomass, decreases in reactor performance in general). Solutions have been proposed, in particular by carrying out cleaning operations with a pause in production, with emptying of the reactor and scraping of the walls.

Patent WO2020/126916 accordingly proposes a technique of cleaning with a basic aqueous solution and steam injection.

Other solutions have been proposed that operate the cleaning without stopping production, which is more advantageous industrially. These relate in particular to the technique described in patents WO 2020/126918 and WO 2020/126917, which propose cleaning of the pretreatment reactor with a basic aqueous solution in the presence of biomass, which, de facto, produces an impregnated and then pretreated biomass that is basic, which can then be followed by the conversion treatment such as enzymatic hydrolysis. However, this basic pretreated biomass is less reactive to enzymatic hydrolysis than biomass impregnated and pretreated in acidic or neutral medium, which is produced outside the cleaning phase, which will tend to mix the two types of pretreated biomass before proceeding with the conversion thereof. This technique is therefore advantageous, but can be improved further so as to make it possible to clean the pretreatment reactor with as little impact as possible on the performance of the pretreatment carried out on the biomass during cleaning.

The aim of the invention is to improve the treatment of lignocellulosic biomass for the purposes of its conversion into sugar(s) or alcohol. More particularly, it aims to increase biomass conversion efficiencies, or to reduce the energy consumption of the treatment or to further make use of any by-products of the treatment.

### SUMMARY OF THE INVENTION

The invention firstly provides a process for treating lignocellulosic biomass, comprising:
b) a step of acidic or neutral pretreatment of the biomass previously placed under acidic or neutral pH conditions in a pretreatment reactor, to produce an acidic or neutral pretreated biomass,
the performance of said step b) alternating with b′) a step of basic pretreatment of the biomass previously placed under acidic, neutral or basic conditions, with optional addition of base, to produce a basic pretreated biomass,
followed by
c) a step of enzymatic hydrolysis in a hydrolysis reactor (16) of the acidic or neutral pretreated biomass obtained at the end of step b) and/or of the basic pretreated biomass obtained at the end of step b′) to obtain a hydrolyzed biomass,
d) a step of solid/liquid separation of the hydrolyzed biomass obtained at the end of step c) in the form of sugar(s) or a step of solid/liquid separation of the hydrolyzed biomass in the form of sugar(s) obtained at the end of step c) then treated in one or more other steps subsequent to the enzymatic hydrolysis step c), in order to obtain a separated juice and an unconverted solid residue,
e) a step of recycling at least part of said unconverted solid residue obtained in step d) to step b′) of basic pretreatment.

For the purposes of the present invention, an unconverted “solid” residue is understood as meaning a residue that comprises at least 20% by weight of solids, in particular at least 30% or 35% by weight of solids. This proportion of solids and soluble compounds in a sample of the product may be evaluated by measuring its dry matter (DM) content, which is measured according to the standard ASTM E1756-08 (2015) “Standard Test Method for Determination of Total Solids in Biomass”.

The invention therefore combines two features:
firstly, it proposes a pretreatment that alternates between acidic or neutral pretreatments for the biomass, which makes it possible to obtain pretreated biomasses that are highly reactive to the enzymatic hydrolysis that follows the pretreatment and the basic pretreatments, which aim to clean the pretreatment reactor while at the same time continuing to produce a basic pretreated biomass that is slightly less reactive (this results in continuous cleaning of the reactor without having to stop the pretreatment),
secondly, it proposes the recycling to the step of basic pretreatment of the biomass of all or part of the unconverted solid residue that is obtained by solid/liquid separation, either after enzymatic hydrolysis (separation on sugar juice) or after fermentation (separation on alcoholic juice), this being introduced specifically to the step of basic pretreatment of the biomass.

It has surprisingly been found that this unconverted solid residue has very advantageous abrasive properties and that its use during the basic pretreatment makes it possible to achieve a cleaning of the walls of the pretreatment reactor that is at least as effective, or even more effective, than with biomass alone.

What is also surprising is that the addition of this residue to the rest of the biomass during treatment did not in fact cause any problems in the operation of the pretreatment tools: it did not complicate the proper functioning of the devices used, whereas it might have been feared that the addition of this residue, having a texture very different from that of the biomass, would lead in particular to problems of fouling of the devices, problems with the conveyance of the residue from one device to another or within a device, etc. That was not the case.

And this combination is very advantageous, with synergies between the two features. Specifically, using this residue to “replace” all or part of the biomass during its basic pretreatment makes it possible to save on biomass during the basic pretreatment and to have more biomass available for the acid pretreatment for the same total mass of biomass to be converted. The effectiveness of the periodic cleaning with basic media of the pretreatment reactor is maintained while producing smaller amounts of basic pretreated biomass that is less reactive, and it is even possible to consider replacing the biomass entirely with this residue during these basic cleaning operations, while at the same time ultimately increasing the conversion efficiency of the biomass to be converted.

And this unconverted solid residue is moreover reused in the production line, which enables any necessary further extraction of any reactive compounds still sequestered in the lignin, for the subsequent steps in the biomass conversion process, which is likewise a move in the direction of increasing biomass conversion efficiency.

The unconverted solid residue thus recycled becomes progressively more enriched in lignin: specifically, the final unconverted residue obtained at the end of production is depleted in the cellulose/hemicellulose that had been sequestered in the lignin and enriched in lignin compared to a residue that has not undergone the recycling according to the invention: this final residue thus has a higher heating value, making it more efficient as a fuel for generating heat via a step of combustion of this final residue. The heat produced can be utilized in the biomass conversion process, in one or more steps requiring heating of a fluid (for example the impregnation liquor), a reactor (for example the cooking during pretreatment) or a reboiler of a distillation column (for example to purify an alcohol obtained by fermentation, etc.). This final unconverted residue can also be made use of outside the biomass conversion line, for uses beyond said line.

It has thus surprisingly been found that reintroducing this residue into the impregnation or cooking device with the biomass during treatment made it possible to:
extract from this residue at least part of the cellulose and/or hemicellulose retained therein, the enzymatic hydrolysis reaction not enabling the complete conversion of these compounds,
and to convert them at least in part by making them follow anew the pathway of the “fresh” biomass in its pretreatment and subsequent enzymatic hydrolysis steps, which in fact ultimately increases the production of sugars (or alcohol) in the process.

As indicated above, step b′) of producing the basic pretreated biomass can in fact be a step of cleaning the impregnation reactor. The pretreatment in neutral or acidic medium will in that case be periodically stopped in particular and the biomass contacted with an alkaline medium in order to allow the residues fouling the walls of the pretreatment reactor to be detached and removed, both through the effect of the alkaline medium capable of dissolving/detaching the residues and through the mechanical friction effect of the biomass, and—in accordance with the invention—of the solid residue that was unconverted in the subsequent steps after the pretreatment.

Pretreatment steps b) and b′) preferably comprise cooking of the biomass, especially cooking accompanied by steam explosion of said biomass.

Advantageously, an amount of unconverted solid residue corresponding to at least 10%, especially at least 20%, preferably at least 30% or 40%, by weight of the total residue and biomass supplied to said pretreatment reactor during said step b′) can in step e) be recycled to basic pretreatment step b′). It is even possible to use even more residue to “replace” the biomass, or to even replace it completely, in this step b′).

The acidic or neutral pretreatment step b) and/or basic pretreatment step b′) preferably includes a prior step a) and/or a′) of impregnation, especially in an impregnation reactor, of the biomass with an acidic, neutral or basic aqueous solution.

And in this case, in a first embodiment, the basic pretreatment step b′) includes a prior step a′) of impregnation, especially in an impregnation reactor, of the biomass with an acidic, neutral or basic aqueous solution, and in step e) at least part of the unconverted solid residue is recycled to pretreatment step b′), said residue being introduced into the impregnation reactor during impregnation step a′). This embodiment is advantageous because it facilitates the extraction of the minority reactive compounds contained in the unconverted solid residue and sequestered therein. However, the presence of this residue in the impregnation reactor can result in additional consumption of impregnation liquor.

In a second embodiment of the invention (with or without prior impregnation in an impregnation device), at least part of the unconverted solid residue can in step e) be recycled to pretreatment step b′), said residue being introduced into the pretreatment reactor, especially a cooking-type reactor, especially with steam explosion. This embodiment is advantageous because the solid residue is carried into the pretreatment reactor by the biomass that has generally undergone prior impregnation with impregnation liquor, and it does not result in any additional consumption of impregnation liquor.

Preferably, step a′) of impregnating the biomass and step b′) of basic pretreatment of the impregnated biomass are carried out in reactors each provided with a biomass feed device, and step e) of recycling the unconverted solid residue is carried out by introducing at least part of said residue with the biomass undergoing treatment into at least one of said feed devices.

“During treatment” is understood as meaning that the residue is introduced before the biomass or with the biomass during at least part of the time the biomass is being fed to the reactor concerned, or after the biomass has been fed to the reactor while the reactor is in operation.

One of the feed devices at least may thus be a feed screw, which is especially at least partly conical, comprising a cowling provided with a cage provided with openings that allow the extraction of the solid-liquid residue from the biomass and optionally the circulation of a wash fluid. This compression screw, as is known, creates a hermetic plug of biomass in the downstream portion of the screw, which creates compression on the biomass that gives rise to a pressure difference between the biomass inlet and the biomass outlet of the screw. But any other known device may be used. For the impregnation step, it is possible to use a reactor operating continuously or in batch mode, or means other than a dedicated reactor, in particular a conveyor belt that is sprayed with liquor, etc.

The basic pretreatment step b′) advantageously comprises an addition of base to the pretreatment reactor: in this case, the biomass is contacted with a basic medium not during its prior impregnation, for example, but during the cooking pretreatment itself. This addition of base into the pretreatment reactor is of course compatible with a prior impregnation of the biomass with an acidic, neutral or basic impregnation liquor: the addition of base is adjusted according to the acidity/basicity of the biomass feeding the pretreatment reactor.

According to one embodiment, the aim of the process according to the invention is the production of sugar juice only, the separation step d) being carried out on the sugar juice exiting the enzymatic hydrolysis.

According to another embodiment, the process aims to convert all or part of the obtained sugar juice by enzymatic hydrolysis. It may then also include:
a step f) of fermenting the hydrolyzed biomass in the form of sugar(s) so as to obtain a fermented biomass comprising at least one alcohol, the solid/liquid separation step d) being carried out on said fermented biomass.

In the latter mode it is possible according to the invention to recycle both the unconverted residue obtained at the end of the enzymatic hydrolysis and/or obtained at the end of the fermentation.

The enzymatic hydrolysis step c) and fermentation step f) can be carried out simultaneously on the pretreated biomass, it is then referred to as SFF, for simultaneous saccharification and fermentation, or SSCF, for simultaneous saccharification and co-fermentation. They can also be carried out one after the other, in particular in separate reactors.

The process according to the invention may also comprise:
a step g) of separation or purification, in particular of distillation, of the fermented biomass, the solid/liquid separation step d) being carried out before or after said separation or purification step g).

Carrying out the separation d) before step g), when step g) is a distillation, is advantageous, since this avoids introducing a liquid phase containing this residue that is capable of fouling the column or at the very least of hampering the operation thereof.

However, it may also prove advantageous to carry out the separation d) of the invention after or during the step of concentration/purification of the hydrolyzate or of the fermentation liquor, since the solid residue may contain a sugar or alcohol fraction and passing it through the concentration/purification step may extract at least part of this fraction of sugar or alcohol sequestered in the solid residue.

As mentioned above, the process according to the invention may also include:
a step h) of combustion of the final unconverted solid residue obtained at the end of the biomass treatment, the heat produced from which is used in a step of said process that requires heating, especially the heating of a fluid, especially in the cooking step b) or b′) or a step g) of separation by distillation.

The final residue obtained with the invention has an improved heating value, due to a lower content of cellulose/hemicellulose, which are polymeric sugars with a low heating value, unlike lignin.

Optionally, the combustion step h) may be preceded by a step i) of drying the residue, in particular so that the residue attains a water content below a given threshold, for example less than or equal to 40% by weight, in particular less than or equal to 30% by weight.

The solid/liquid separation step d) may be carried out by filtration, in particular using a pressing or draining device, such as a filter press or a vacuum filter, a belt filter, a belt press or a centrifuging, decanting or dewatering device or the combination of different devices.

The unconverted solid residue obtained in the solid/liquid separation step d) generally contains, in addition to water, a majority compound in the form of lignin, and minority compounds including cellulose and/or hemicellulose and possibly one or more ethanol-type alcohols when the solid/liquid separation d) is carried out after a fermentation step f).

The unconverted solid residue obtained in the solid/liquid separation step d) may contain, by way of example, between 40% and 70% by weight of water, in particular between 50% and 60% by weight of water, between 2% and 35% DM of cellulose, in particular between 5% and 20% DM of cellulose, and between 0% and 15% DM of hemicellulose, in particular between 1% and 10% DM of hemicellulose. It may also include other minority compounds, such as mineral compounds (which can be measured by analyzing their ash after combustion).

The different contents of lignin, cellulose, and hemicellulose can vary, in particular according to the type of biomass used and according to the reactivity thereof in the enzymatic hydrolysis in particular.

Step e) of recycling the unconverted solid residue decreases its content, in the final solid residue obtained at the end of the biomass treatment, of at least one of its cellulose and hemicellulose minority compounds and increases its heating value, which is beneficial both for the biomass conversion efficiency and for the thermal integration of the process.

The dry basis composition of the final solid residue obtained at the end of the biomass treatment generally comprises mainly lignin, and less than 20% DM of cellulose and less than 8% DM of hemicellulose.

The treatment process that is the aim of the invention can convert the lignocellulosic biomass into sugar juice, in particular C5 and C6 juices, after enzymatic hydrolysis, or into alcohol(s), in particular ethanol, after fermentation of said sugar juice.

The invention also relates to any plant performing the process described above.

The invention further provides the plant for performing the process, which comprises:
a) a device, in particular a reactor, for impregnating the biomass with an aqueous solution, in particular an acidic or neutral solution,
b) a reactor for pretreatment by cooking of the impregnated biomass originating from the impregnation device, said cooking optionally being accompanied by steam explosion, to obtain a pretreated biomass, said reactor being provided with means for injecting basic aqueous solution,
c) a reactor for enzymatic hydrolysis of the pretreated biomass to obtain a hydrolyzed biomass, such that said plant also comprises:
d) a device for solid/liquid separation of the hydrolyzed biomass or of the hydrolyzed biomass subsequently treated in one or more other reactors or devices arranged downstream of the enzymatic hydrolysis reactor c), in order to obtain an unconverted solid residue,
e) means for recycling at least part of said unconverted solid residue in the impregnation reactor a) to the impregnation reactor and/or to the pretreatment reactor when the pretreatment reactor is operated under basic conditions.

The means for recycling the unconverted solid residue are conventional, and may include any suitable system of conveyance (for example by conveyor, screw, hopper or belt).

Preferably, the plant according to the invention is such that the impregnation device a) and the pretreatment reactor b) are provided with feed devices, and such that the recycling means e) comprise fluid connection means between the solid/liquid separation device d) and at least one of said feed devices for conveying the unconverted solid residue from the separation device to said feed device or to at least one of said feed devices.

The invention further provides for the use of the process or of the plant described above for the treatment of lignocellulosic biomasses, such as wood, straw, agricultural residues, paper-making residues, and all dedicated energy crops, in particular annual or perennial plants such as miscanthus, with a view to producing sugars, alcohol-type biofuels or biobased molecules.

The invention will be described below in greater detail with the aid of the figures and nonlimiting examples.

### LIST OF FIGURES

FIG. 1 is a schematic representation of a first variant of a plant for converting lignocellulosic biomass where the process according to the invention may be applied.

FIG. 2 is a schematic representation of a second variant of a plant for converting lignocellulosic biomass where the process according to the invention may be applied.

FIG. 3 is a schematic representation of a third variant of a plant for converting lignocellulosic biomass where the process according to the invention may be applied.

FIG. 4 is a schematic representation of a plant for converting lignocellulosic biomass applying the invention.

It should be noted that the same references relate to the same stream and/or same device from one figure to the next.

The description of the references is presented below:
1: Water inlet into the liquor preparation tank
2: Acid inlet into the liquor preparation tank
3: Liquor preparation tool (tank)
4: Acidic liquor to impregnation tool (reactor)
5: Ground biomass
6: Impregnation tool feed device
7: Wash water of the impregnation tool feed device
8: Wash liquid outlet of the impregnation tool feed device
9: Impregnation tool (reactor)
10: Impregnated and drained biomass
11: Pretreatment tool feed device
12: Wash water of the pretreatment tool plug-screw feeder
13: Pressate of the pretreatment tool feed device
14: Pretreatment cooking tool (explosion reactor)
15: Injection of steam for the pretreatment
16: Pretreated biomass and steam
17: Tool (cyclone) for separating steam and pretreated biomass
18: Steam
19: Pretreated biomass
20: Enzymatic hydrolysis reactor
21: Hydrolysate containing sugars
22: Alcoholic (ethanolic) fermentation reactor
23: Fermentation liquor containing ethanol (alcohol)
24: Ethanol recovery device, for example distillation column(s)
25: Concentrated alcohol
26: Liquid residue (crude vinasses)
27: Solid (lignin)/liquid separation, for example a filter press
28: Liquid residue (vinasses)
29: Unconverted solid residue (lignin cake)
30: Clarified (solid-free) fermentation liquor
31: Clarified (solid-free) hydrolyzate

### DESCRIPTION OF EMBODIMENTS

The invention proposes to recycle the unconverted solid residue from the process for converting lignocellulosic biomass into sugars or alcohols. This residue is mainly composed of lignin, a compound that is not converted during the process. This residue is usually separated in order to be burned in conventional biorefineries, the lignin can also be used as a compound of interest for specialty products (resins, bitumen, etc.). The invention proposes to recycle this compound upstream in the process, because the extracted lignin is not pure: it contains a fraction of polymeric sugars (cellulose and/or hemicellulose) that is not converted. Recycling these sugar polymers then makes it possible to increase the yield of the process.

The invention relates to the recycling of at least part of a solid residue referred to in the present text as “lignin cake” or “unconverted solid residue”. This solid is advantageously recycled to the step of pretreatment under basic conditions of the lignocellulosic biomass, which here also serves as a step of cleaning the pretreatment reactor, which is a cooking reactor with steam explosion of the biomass.

The preferred embodiment of the invention will be described here in which the pretreatment by explosive cooking is preceded by impregnation of the biomass with an acidic liquor: periodically biomass is transferred to an alkaline medium in the cooking reactor in order to clean the reactor.

Lignin is one of the majority compounds of lignocellulosic biomass (up to 30% DM). In a lignocellulosic biomass conversion process making use of the sugars (cellulose and hemicellulose), the lignin cake is a co-product of the process that is composed of water (water can represent 50% to 60% by weight of the cake) and solids originating from the biomass that are not converted during the process, namely lignin mainly, but also cellulose and hemicellulose.

Thus, Table 1 below, by way of example, indicates the throughput entering the process and the composition of a typical example of biomass (wheat straw) to be treated and of a lignin cake obtained after separation at the end of the alcohol production line:

TABLE 1

| | | Biomass | Unconverted solid |
|---|---|---|---|
| Throughput | kg/h | 100 | 67 |
| Water | wt % | 10 | 60 |
| Cellulose | wt % | 34 | 8 |
| Hemicellulose | wt % | 27 | 3 |
| Lignin | wt % | 15 | 22 |
| Others | wt % | 14 | 7 |

And Table 2 below indicates, for the same biomass and the same lignin cake, their throughputs and composition of the solid compounds, expressed here as content of dry matter, DM: Table

| | | Biomass | Unconverted solid |
|---|---|---|---|
| Throughput | kg/h | 100 | 67 |
| DM | wt % | 90 | 40 |
| Cellulose | % DM | 38 | 20 |
| Hemicellulose | % DM | 30 | 8 |
| Lignin | % DM | 17 | 55 |
| Others | % DM | 16 | 17 |

It can be seen from these tables that two unconverted sugar polymers (cellulose and hemicellulose) are present in the lignin cake, for a total of 11 wt % (or 28% DM). The invention recycles at least part of the lignin cake (unconverted solid) to convert these polymers and thus increase the yield of the process, given that 11% of the polymeric sugars of the biomass are present in the unconverted solid, without adding a treatment step dedicated to this lignin cake.

In addition, the heating value of the lignin cake (measured by the higher heating value HHV of the unconverted solid residue) increases on conversion of the sugar polymers present therein. This is because the sugar polymers contain oxygen molecules that reduce the average HHV of the solid.

Examples of operating conditions for key steps of the biomass treatment are briefly summarized below:

The process:

Below is a more detailed description of the various key steps of a biomass conversion process using such a plant and to which the invention may advantageously be applied: (this is one example; the invention is not limited thereto).

Step of conditioning the lignocellulosic biomass:

The treatment process may conventionally comprise in its first step a step of conditioning the lignocellulosic biomass with at least one grinding operation so as to obtain biomass particles having a size of at most 300 mm. It is of course possible to carry out two or more successive grinding steps in order to attain the target particle size. In general, the ground biomass has a particle size (largest size) of at most 300 mm, usually of at least 1 mm, and commonly between 2 and 200 mm. Any method known to those skilled in the art can be used to carry out this step. Usually, straw is ground using screens of from 5 to 100 mm. In the case of wood, this is generally chipped into parallelepipedal chips with a length of between 20 and 160 mm, a width of between 10 and 100 mm, and a thickness of between 2 and 20 mm. The ground lignocellulosic biomass is conveyed to the next step by any means known to those skilled in the art, in particular a screw conveyor.

Step of impregnation with an acidic liquor:

The treatment process according to the invention comprises a step a) of impregnating the lignocellulosic substrate with an acidic liquor, so as to obtain an impregnated lignocellulosic substrate having a pH of from 0.1 to 3. The aim of this step is to prepare the lignocellulosic substrate for the pretreatment step.

The impregnation is carried out in an impregnation reactor at a temperature of between 10° C. and 90° C. and preferably at atmospheric pressure. The residence time of the lignocellulosic substrate in the impregnation reactor is usually from 10 seconds to 180 minutes, preferably between 30 seconds and 60 minutes, and even more preferably between 30 seconds and 15 minutes. The impregnation step is preferably performed in a single step.

The impregnation reactor or impregnator is provided with one or more screws that transfer the lignocellulosic substrate from its inlet to the outlet opening. The impregnator is moreover provided with one or more conduits for conveying the acidic liquor and also, if required, one or more conduits for withdrawing acidic liquor. Said acidic liquor inlet and outlet conduits are generally installed so as to function by cocurrent or counter-current recycling.

The acidic liquor is an aqueous solution of a strong acid, which is for example selected from sulfuric acid, hydrochloric acid, and nitric acid having for example an acid content of between 0.5% and 4% by weight.

Step of solid/liquid separation on the lignocellulosic substrate impregnated with acidic liquor: The lignocellulosic substrate impregnated with acidic liquor is subjected to a solid/liquid separation step in order to obtain a lignocellulosic substrate having a dry matter content of between 15% and 70% by weight and a used acidic liquor. The lignocellulosic substrate impregnated with acidic liquor is preferably first drained, in order to extract at least part of the free acidic liquor, before being treated by solid/liquid separation.

The solid/liquid separation step may employ any technique known to those skilled in the art, which may be for example decantation, centrifugation or pressing.

Pressing of the lignocellulosic substrate is preferably carried out concomitantly with its transfer to the pretreatment step when the latter employs the steam explosion process described below. This method of carrying out the step is for example performed by a compression screw termed a “plug screw feeder”, the operation of which has already been described above. The formation of a plug of pressed lignocellulosic substrate ensures the pressure-tightness of the steam explosion reactor, thus preventing dangerous escapes of steam. The screw conveyor is also provided with one or more conduits for withdrawing the used liquor (referred to as pressate) separated during pressing. The pressate can be recycled to the impregnation step and/or to the step of washing with the wash liquid 12 passing through the feed screw 11.

The wet biomass obtained at the end of the solid/liquid separation step, which can be denoted by the term “washed and acidified lignocellulosic substrate” has a dry matter content of preferably between 15% and 70% by weight, and more preferably between 40% and 65% by weight.

Step of pretreating the washed and acidified lignocellulosic substrate:

The washed and acidified lignocellulosic substrate undergoes a pretreatment step.

The cellulose (and optionally hemicelluloses) that are the targets of the enzymatic hydrolysis are not directly accessible to the enzymes. This is the reason why a pretreatment of the biomass is carried out before the enzymatic hydrolysis step. The pretreatment is aimed in particular at modifying the physical and physicochemical properties of the cellulosic fraction, such as its degree of polymerization and its state of crystallinity.

Various types of pretreatment are known to those skilled in the art, they combine a chemical treatment and a heat treatment. These include, in particular, acidic or basic cooking, the organosolv process, treatments with ionic liquids, and the steam explosion process.

The preferred pretreatment process is steam explosion (or “SteamEx”) carried out in an acidic medium. This is a process in which the lignocellulosic substrate is rapidly brought to a high temperature by injecting pressurized steam. Stoppage of the treatment is achieved by abrupt decompression.

The operating conditions of the steam explosion process are as follows:
steam is injected directly into the reactor,
the temperature of the reactor is generally between 150° C. and 220° C., preferably between 170° C. and 210° C.,
the pressure is between 5 and 25 bar absolute (0.5 and 2.5 MPa), more preferably between 8 and 19 bar absolute (0.8 to 1.9 MPa),
the residence time before the expansion phase ranges from 10 seconds to 90 minutes and preferably between 3 minutes and 30 or 40 minutes.

The steam explosion may be performed in batch or continuous mode and the depressurization step that permits destructuring of the biomass may proceed in one or more steps.

At the end of the steam explosion pretreatment step, a pretreated lignocellulosic substrate with a high dry matter content, generally between 20% and 70% by weight, and a vapor phase which may be condensed, are obtained.

Following the steam explosion under acidic conditions, the pretreated lignocellulosic substrate generally has a pH lower than is compatible with the medium for the enzymatic hydrolysis. The lignocellulosic substrate is accordingly subjected to a neutralization step to bring its pH to a value between 4 and 6.

For the neutralization step, an aqueous solution is used that contains a neutralizing agent which may be selected from all weak or strong bases known to those skilled in the art. The term base denotes any chemical species that, when added to water, gives an aqueous solution with a pH of greater than 7. Preferably, the neutralizing agent is selected from potassium hydroxide, sodium hydroxide, aqueous ammonia and lime. Even more preferably, the neutralizing agent is selected from potassium hydroxide and aqueous ammonia, alone or in combination with one another. Preferably, the neutralizing agent is used in aqueous solution, with a concentration by weight of between 2% and 75%, and even more preferably between 20% and 70%.

Neutralization is carried out at a temperature of between 15° C. and 95° C., and preferably between 20° C. and 70° C. In general, the temperature of the neutralization step is not precisely controlled and is simply governed by the heat given off by the acid-base neutralization reaction. The neutralization step can be carried out continuously, in batch mode or in fed-batch mode. It should be noted that an optional washing step can be carried out before or after the neutralization step, on all or part of the pretreated lignocellulosic substrate.

If a washing is applied, a liquid stream is contacted with the pretreated lignocellulosic substrate, then the liquid is separated from the solid. The washing step can be carried out by percolation, by successive mixing and liquid/solid separation operations, by washing on a belt filter or by any other technique known to those skilled in the art. The wash liquid used can be water or a process stream. The weight ratio between the wash liquid added and the liquid contained in the substrate to be washed is generally between 0.5 and 4. The washing step generates a sugary wash liquor containing part of the hemicelluloses solubilized during the pretreatment. This wash liquor can for example be used as a source of carbon for the production of biocatalysts (enzymes and/or microorganisms). The washing step is generally carried out at a temperature of between 10° C. and 95° C.

Enzymatic hydrolysis step:

The pretreated lignocellulosic substrate, optionally neutralized and washed, is sent to the enzymatic hydrolysis step of the process.

The pretreated lignocellulosic substrate sent to the enzymatic hydrolysis step has a dry matter content generally of between 15% and 70% by weight.

The objective of the enzymatic hydrolysis is to hydrolyze (depolymerize), by means of biocatalysts, the hemicelluloses and cellulose into fermentable sugars, preferably xylose and glucose.

The enzymatic hydrolysis step is carried out under mild conditions, at a temperature of the order of 40° C. and 55° C., preferably between 45° C. and 50° C. and at a pH of from 4.0 to 5.5, and even more preferably between 4.5 and 5.2. The dry matter content of the enzymatic hydrolysis medium is between 2% and 45% by weight, preferably between 10% and 30% by weight. It is carried out by means of enzymes produced by a microorganism. Natural or genetically modified microorganisms, such as fungi of the genera Trichoderma, Aspergillus, Penicillium or Schizophyllum, or anaerobic bacteria of, for example, the genus Clostridium, produce a cocktail of enzymes containing in particular cellulases and hemicellulases suitable for extensive hydrolysis of cellulose and hemicelluloses.

The enzymatic hydrolysis can be carried out continuously or in batch mode or in fed-batch mode, in one or more reactors. The residence time is between 5 hours and 200 hours and preferably between 24 hours and 120 hours and even more preferably between 48 hours and 120 hours.

At the end of the step, a hydrolyzate containing fermentable sugars is recovered from the bioreactor and is then treated in the fermentation step.

It should be noted that the hydrolyzate obtained can optionally undergo one or more treatment steps before the fermentation step. For example, there may be a pH adjustment, a partial purification with a view to limiting the content of inhibitor compound for the fermentation microorganism, or an at least partial separation of the solid residues contained in the hydrolyzate (and thus to obtain the unconverted solid residue to be treated according to the invention).

Step of fermenting the hydrolyzate, when this is to be followed by conversion of the sugars obtained into alcohol(s):

In the step of the process for producing solvents and/or alcohols, the optionally treated hydrolyzate is sent to the fermentation step enabling the conversion, by one or more microorganisms of various genera, of the fermentable sugars into solvent and/or alcohols of interest. The fermentation methods are known to those skilled in the art and are described in particular in document U.S. Pat. No. 8,456,633.

The term “solvent” is intended to denote organic compounds other than alcohols, for example organic compounds having a ketone function such as acetone.

The term “alcohol” denotes in particular ethanol, propanol, isopropanol, and butanol.

The natural or genetically modified microorganisms can be selected for example from Saccharomyces cerevisiae, Schizosaccharomyces pombe, Saccharomyces uvarum, Saccharomyces diastaticus, Kluyveromyces fragilis, Candida shehatae, Pichia stipitis, Pachysolen tannophilis or the bacteria Zymomonas mobilis, Clostridium acetobutylicum, and Escherichia coli.

In the context of the invention, the fermentation step makes it possible, for example, to produce ethanol alone or as a mixture with butanol, propanol, isopropanol and/or acetone. For example, the fermentation microorganism may be capable of producing an “ABE (acetone-butanol-ethanol)” mixture or else an “IBE (isopropanol-butanol-ethanol)” mixture.

The microorganism selected is preferably a natural or genetically modified yeast of the genus Saccharomyces capable of producing ethanol.

At the end of the step, a fermentation must diluted in products of interest is recovered.

According to one embodiment of the process, the hydrolysis and fermentation steps may be carried out at the same time, in at least one same bioreactor, so that the enzymatic hydrolysis and the fermentation are carried out simultaneously according to a process denoted by the term “simultaneous saccharification and fermentation (SSF)”. When the hydrolysis step is merged with the fermentation step, the operating conditions, in particular temperature conditions, can be adapted to be compatible with the tolerances of the fermentation microorganism. For example, the temperature can be lowered to between 28° C. and 45° C., and preferably between 30° C. and 35° C., when the fermentation is carried out with a yeast of the genus Saccharomyces. The pH is preferably adjusted to between 5 and 5.5 in order to favor the performance of the yeasts.

The production unit carrying out the process according to the invention may comprise, in addition to the plants already described, units for the in-situ production of enzymes and/or yeasts.

Step of concentrating and purifying the solvents and/or alcohols from the fermentation must: The process according to the invention lastly comprises a step of concentrating and purifying the product(s) of interest from the fermentation must, which is preceded or followed by a solid/liquid separation step in order to remove at least a fraction of the solid matter contained in the fermentation must, and to produce the unconverted solid residue that is to be treated according to the invention.

The step of concentrating and purifying the product(s) of interest, for example ethanol, preferably employs one or more distillations, which is technology well known to those skilled in the art.

The feedstock: lignocellulosic biomass:

In accordance with the invention, the feedstock of the process may be a biomass alone or as a mixture. The amount of water contained in the crude feedstock is generally at least 10%, especially between 10% and 70% by weight.

The crude biomass is selected from any type of biomass, preferably biomass of solid type, and in particular biomass of lignocellulosic type. Nonlimiting examples of biomass types include for example farming residues (especially straw, corn cobs), forestry residues, forestry products, sawmill residues, and dedicated crops, for example short-rotation coppice.

Preferably, the crude biomass, also referred to as native biomass, is lignocellulosic biomass. It essentially comprises three natural constituents present in variable amounts depending on its origin: cellulose, hemicellulose, and lignin.

The lignocellulosic biomass feedstock is preferably used in its crude form, i.e. containing all three of the constituents cellulose, hemicellulose, and lignin.

In a preferred embodiment of the invention, the lignocellulosic biomass is selected from grassy biomass, farming residues such as straw waste, corn cobs, sugarcane bagasse, forestry residues or sawmill residues such as wood chips or any other type of wood residues.

The impregnation fluid:

The optional fluid injected for the impregnation is an aqueous liquid solution optionally containing acid, at a temperature between 10° C. and 95° C. and at atmospheric pressure. The pH of this chemical solution is between 0.1 and 12.0, preferably between 0.1 and 7, preferably between 0.3 and 2. According to a preferred embodiment, the liquor used is an acid catalysis liquor, and the pH of the liquor is adjusted to between 0.1 and 4, especially between 0.3 and 2. The acid used may for example be at least one acid selected from sulfuric acid, hydrochloric acid, nitric acid, and oxalic acid. Their content in the aqueous phase is preferably between 0.2% and 8% by weight.

The invention similarly applies to different processes and plants, in particular to:
plants aimed solely at producing sugars, which therefore have no provision for alcoholic fermentation,
plants with provision for a cooking pretreatment without prior impregnation by a liquor (autohydrolysis for example),
plants with provision for pretreatment by cooking with prior impregnation with a neutral liquor (with in particular autohydrolysis of the biomass in contact with water, naturally releasing an acid, in particular acetic acid)

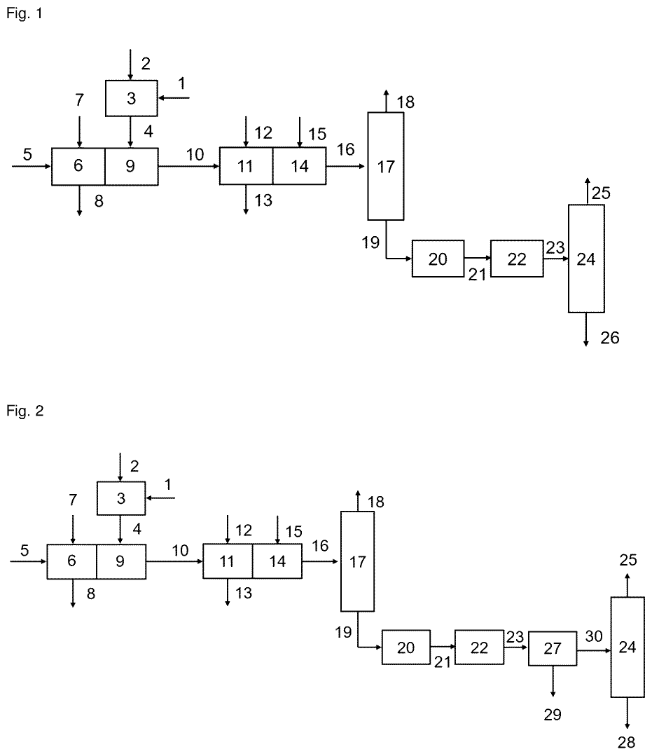

FIG. 1 represents in very schematic form a nonlimiting variant of a plant for converting lignocellulosic biomass in which the invention can be applied; this plant has provision for pretreatment of the biomass including an impregnation with an acidic liquor, then cooking/steam explosion, then enzymatic hydrolysis followed by alcoholic fermentation to convert the biomass to ethanol.

FIG. 1 therefore represents a biomass conversion carried out in the following manner: the ground biomass 5 (optionally impregnated with a catalytic liquor) is introduced into an impregnation reactor 9 via a feed device 6. This may be a pressurized feed means, for example a compression feed screw, the end portion of which is conical, which has a cowling with a drainage screen, a wash water inlet 7, and a wash water outlet 8. A hermetic plug of biomass is created in the downstream portion of the screw, which creates compression on the biomass that gives rise to a pressure difference between the biomass inlet and the biomass outlet of the screw of at least 0.05 MPa. The compression applied to the biomass may result in expulsion of part of the liquid contained in the biomass, particularly when the DM of the biomass is less than 80% before it enters the pressurized feed means 6. The liquid thus extracted mixes with the wash water and is drawn off with the used wash water 8.

The impregnation reactor 9 is likewise supplied with acidic liquor 4 (water with sulfuric acid added) originating from a liquor preparation tank 3, itself supplied with sulfuric acid 2 and with water 1.

The impregnated and drained biomass 10 exits the reactor 9 to feed a steam explosion cooking reactor 14 via another feed device 11, for example of compression type like the feed device 6. In this device 11, owing to the compression exerted on the biomass, a liquid 13 resulting from this pressing, also referred to as pressate, is recovered, which is composed of water and acid. The device 11 is washed, using a dedicated inlet, with a wash liquid 12 (water and/or a recycled liquid, as seen below), which is then discharged via the outlet through which the pressate 13 is also discharged.

The reactor 14 is also supplied with steam 15. On exiting the reactor 14, the biomass-steam mixture passes into a tool 17 for separating the biomass 19 and the steam 18. The biomass 19 is then treated in an enzymatic hydrolysis reactor 20, then once it has been hydrolyzed to sugars, the hydrolyzed biomass 21 (also referred to as hydrolysis must) passes into an alcoholic fermentation reactor 22. The biomass fermented to alcohol 23 (also referred to as fermentation must) is then conveyed into one or more distillation columns 24 to obtain concentrated alcohol 25 and crude vinasses 26, which are solid/liquid residues, as a mixture or separated depending on the arrangement of the columns 24.

A solid/liquid separation is carried out by a device of filter press type (not depicted), affording at the outlet a liquid residue not depicted (the vinasses) and a residue not depicted that is the lignin cake of interest in the invention.

This is just one example of a plant, which can also have numerous variants. Thus, the enzymatic hydrolysis and the fermentation may be carried out together in the same reactor; it is then referred to as SSCF (simultaneous saccharification and co-fermentation).

FIG. 2 is a variant of the process shown in FIG. 1 in which, everything else being the same, the solid/liquid separation by means of a filter press 27 is performed on the fermentation liquor 23 upstream of the distillation column(s) 24; a lignin cake 29 is extracted and the liquid residue, that is to say the fermentation liquor without solid residue, 30 is then conveyed into the distillation column(s) 24.

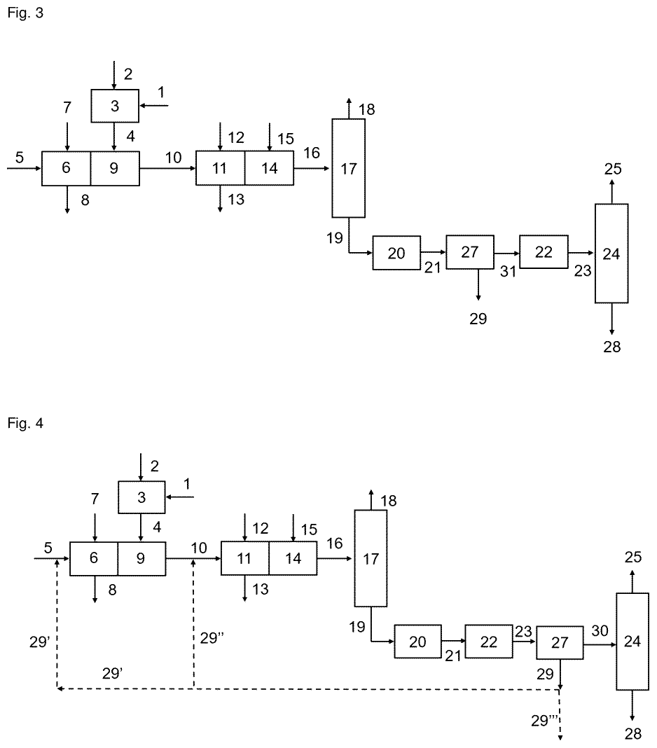

FIG. 3 is a variant of the process shown in FIG. 2 in which, everything else being the same, the solid/liquid separation device 27 of filter press type is arranged between the enzymatic hydrolysis reactor 20 and the fermentation reactor 22; in this case the separation is carried out on the hydrolyzate 21 which is not yet fermented, and the liquid portion of the hydrolyzate (sugar juice) 31 then goes into the fermentation reactor 22.

In a variant not depicted of the process shown in FIG. 3, the aim is solely the production of sugar juice 31, without following this with conversion by fermentation on the same production line.

In all these variants, the objective of the invention is to treat the lignin cake 29, which is ultimately intended to be burned to produce energy.

FIG. 4 shows two alternative or cumulative implementations of the invention, starting from the variant of FIG. 2:

The invention will recycle this solid residue, this lignin cake, during the phase of cleaning the cooking reactor 14 by adding base (KOH) to the cooking reactor 14.

This cooking of the biomass in a basic medium, alternating with cooking of the acid-impregnated biomass, serves for the periodic cleaning of the cooking reactor in accordance with the technique described in the aforementioned patents WO 2020/126918 and WO 2020/126917.

To give an example of this alternating of pretreatment under acidic and basic conditions, it is possible for example to operate the cooking reactor 14 in two alternate modes:
under acidic conditions, with impregnation with an acidic liquor in the impregnation reactor for 100 hours, then transfer to the cooking reactor 14,
under basic conditions, with direct injection of a base into the cooking reactor 14, in particular through a dedicated inlet. Alternatively, the base may also, at least in part, be injected into the cooking reactor before feeding the unconverted wood residue (lignin) into the reactor. One possibility is to feed the cooking reactor with exclusively this unconverted wood residue, via the impregnation device upstream of the cooking reactor. In this case, it may not be necessary to inject a base into the cooking reactor; it is also possible to cut off the acid feed to the impregnation device upstream of the cooking reactor or to perform a basic impregnation.

Implementation of the invention by recycling the lignin cake 29 to the feed device 6 of the impregnation reactor 9:

This recycling before impregnation is advantageous because the lignin cake 29 (stream 29′ in FIG. 4) will have renewed contact with the (acidic) impregnation liquor in the reactor 9, which will help to extract the residual polymeric sugars contained in the cake and subsequently make them react more easily during the enzymatic hydrolysis. One concern might have been that the lignin cake would crumble and settle at the bottom of reactor 9 without being able to be carried with the biomass. However, this was surprisingly not the case, even when the biomass is carried within the reactor from its injection point to its exit point by means of screws inside the reactor.

Implementation of the invention by recycling the lignin cake 29 to the feed device 11 of the cooking reactor 14:

The lignin cake is recycled at least in part (stream 29″ in FIG. 4) to the feed device 11 of the cooking/steam explosion reactor 14. In this case, the lignin cake does not undergo renewed impregnation with liquor, but is carried by the biomass that has already been impregnated.

In either embodiment, part of the lignin cake (stream 29″ in FIG. 4) is optionally not recycled: The lignin cake may be only partially recycled.

At the end of production, the unrecycled cake 29″ (the residue termed the final residue) is stored to serve as fuel, either on the production line or for another use outside the production line, as fuel or to be incorporated into various products.

The same types of recycling similarly apply to the lignin cakes according to the processes shown in FIGS. 1 and 3.

### EXEMPLARY EMBODIMENTS

### Example 1 (Comparative)

In this example, which is not in accordance with the invention, the unconverted solid 29 is separated without being recycled.

The biomass 5 is a lignocellulosic biomass: wheat straw. The characteristics and composition of the wheat straw feedstock are as follows:
Dry matter: 91.07%
Potential sugar: 67.5 g/100 g
Biomass throughput: 65 kg DM/h
Particle size: 50 mm

The biomass is treated according to the process shown in FIG. 2. After hydrolysis, the cellulose is converted into glucose or into glucose oligomers, and the hemicellulose is converted into xylose or into xylose oligomers. For this, the cellulose or hemicellulose throughputs are expressed as potential glucose or potential xylose.

The term “potential sugar” (xylose, glucose for example) used below defines the addition of the various sugars irrespective of their form: monomeric or polymeric sugar and corresponds to the theoretical amount of sugar that can be produced from the composition of the raw material, in this case biomass. Specifically, after pretreatment by cooking, part of the sugars remains in the form of sugar polymers (cellulose or hemicellulose for example), and part of the sugars is in the form of sugar monomers (glucose or xylose for example). This measurement may be carried out using the standard ASTM E1758-01 (2020) “Standard Test Method for Determination of Carbohydrates in Biomass by High Performance Liquid Chromatography” As defined in the standard, to express this amount of sugars in polymer form (cellulose for example), it is necessary to subtract the water of hydrolysis from this amount.

The operating procedure is as follows:

The operating conditions are described in detail below:
Mode 1=production:
   Impregnation for production in the impregnation reactor 9:
Acidic solution flow rate: 1.5 kg/h (H₂SO₄)
Impregnation temperature: 80° C.
   Steam explosion of the impregnated biomass in the pretreatment reactor 14:
Residence time: 5 min
Steam temperature: 190° C.
Production time: 20 hours

After 20 hours of production, the cleaning sequence is carried out under the following conditions:
Mode 2=production+cleaning:
   Impregnation in the impregnation reactor 9:
Acidic solution flow rate: 1.5 kg/h (H₂SO₄)
Impregnation temperature: 80° C.
   Steam explosion of the impregnated biomass in the pretreatment reactor 14:
Residence time: 10 min
Temperature in the reactor: 200° C.
KOH temperature: 130° C.
KOH flow rate: ~300 kg/h Cleaning time: 2 hours
Concentration of KOH used in the liquor: 7.7% by weight
Number of cycles: 3 cycles of mode 1 (production) and 1 cycle of mode 2 (production+cleaning)
   Cleaning of the cyclone 17 (separation device)
Number of water flushes: 2, after each cleaning operation

After 3 cycles of mode 1+mode 2, for a total production time of 66 h (60 hours of production and 6 hours of cleaning), the cleaning was found to be effective.

The mass of material cleaned from the reactor at the end of the procedure (i.e. after these 3 cycles) does not exceed 3 kg. The completion of a cycle required:
1300 kg of biomass for production (20 h)
130 kg of biomass for production and cleaning (2 h) to produce 325 kg of ethanol with a DM yield of 22.7%

### Example 2 (According to the Invention)

This example according to the invention proposes recycling the unconverted solid 29 to the inlet of the cooking reactor 14 via the feed screw 11 in the cleaning phase. The biomass used for this example is the same as for example 1.

The biomass is treated according to the process shown in FIG. 2. After hydrolysis, the cellulose is converted into glucose or into glucose oligomers, and the hemicellulose is converted into xylose or into xylose oligomers.

The operating conditions are described in detail below:
Mode 1=production:
   Impregnation for production in the impregnation reactor 9:
Feedstock: Wheat straw at a throughput of 65 kg DM/h
Acidic solution flow rate: 1.5 kg/h (H₂SO₄)
Impregnation temperature: 80° C.
   Steam explosion of the impregnated biomass in the pretreatment reactor 14:
Residence time: 5 min
Steam temperature: 190° C.
Production time: 20 hours

After 20 hours of production, the cleaning sequence is carried out under the following conditions:
Mode 2=production+cleaning: (100% recycling of lignin residue 29)−Impregnation in the impregnation reactor 9:
Feedstock: 65 kg DM/h of recycled lignin (=unconverted wood residue 29)

The acid feed to the impregnation reactor is stopped.
Impregnation temperature: 80° C.
   Steam explosion of the impregnated biomass in the pretreatment reactor 14:
Residence time: 10 min
Temperature in the reactor: 200° C.
KOH temperature: 130° C.
KOH flow rate: ~300 kg/h
Cleaning time: 2 hours
Concentration of KOH in the liquor: 7.7% by weight
Number of cycles: 3 cycles of mode 1 (production) and 1 cycle of mode 2 (production+cleaning)
   Cleaning of the cyclone 17 (separation device)
Number of water flushes: 2, after each cleaning operation

After 3 cycles of mode 1+mode 2, for a total production duration of 66 h (60 hours of production and 6 hours of cleaning).

The completion of a cycle required:
1300 kg of biomass for production (20 h)
0 kg of biomass for production and cleaning (2 h) replaced by recycled lignin
   to produce 325 kg of ethanol with a DM yield of 25.0%

This example shows that when replacing the biomass with lignin (the unconverted residue) at a content of 100%, the cleaning was found to be as effective as in example 1. Biomass consumption also decreased in the production of basic marc, with a 100% reduction during the cleaning phase, which represents an increase in ethanol yield of 9% over the duration of the cycle.

In conclusion, compared with the prior art, the present invention leads to the following advantages:
Cleaning of the cooking reactor in alkaline medium is at least as effective when all or part of the biomass is replaced by the unconverted solid residue (lignin cake 29).
The overall sugar/ethanol yield of the process can also be improved by recycling the unconverted polymeric sugars (cellulose and hemicellulose) contained in this wood residue. After recycling to the pretreatment, these polymeric sugars are partly converted into monomeric sugars or are made more accessible to the enzymes used in the enzymatic hydrolysis step. In any case, even if the celluloses and hemicelluloses sequestered in the lignin are not extracted by this recycling, in particular when recycling takes place to the cooking reactor rather than to the impregnation reactor, the overall yield is improved by the fact that the same amount of alcohol/sugars is produced with less biomass, the lignin cake replacing part of the biomass during the basic cooking in the reactor 14.
The quality of the lignin of the final residue is improved, since the conversion of the sugars will have made it purer/richer in lignin; the HHV of the solid is thus increased, since the polymeric sugars have an HHV lower than that of lignin.
Less biomass is “consumed” during the cleaning phase in basic medium of the cooking reactor 14, since all or part of this material can be replaced by lignin cake, which makes it possible to produce more sugars/alcohol for the same amount of biomass.

1. A process for treating lignocellulosic biomass, comprising:
b) a step of acidic or neutral pretreatment of the biomass previously placed under acidic or neutral pH conditions in a pretreatment reactor, to produce an acidic or neutral pretreated biomass,
the performance of said step b) alternating with
b′) a step of basic pretreatment of the biomass previously placed under acidic, neutral or basic conditions, in the pretreatment reactor, with optional addition of base, to produce a basic pretreated biomass,
followed by
c) a step of enzymatic hydrolysis in a hydrolysis reactor (16) of the acidic or neutral pretreated biomass obtained at the end of step b) and/or of the basic pretreated biomass obtained at the end of step b′), to obtain a hydrolyzed biomass,
d) a step of solid/liquid separation of the hydrolyzed biomass obtained at the end of step c) in the form of sugar(s) or a step of solid/liquid separation of the hydrolyzed biomass in the form of sugar(s) obtained at the end of step c) then treated in one or more other steps subsequent to the enzymatic hydrolysis step c), in order to obtain a separated juice and an unconverted solid residue,
e) a step of recycling at least part of said unconverted solid residue obtained in step d) to step b′) of basic pretreatment.

2. The process as claimed in claim 1, characterized in that step b′) of producing the basic pretreated biomass is a step of cleaning the impregnation reactor.

3. The process as claimed in claim 1, characterized in that pretreatment steps b) and b′) comprise cooking of the biomass, especially cooking accompanied by steam explosion of said biomass.

4. The process as claimed in claim 1, characterized in that an amount of unconverted solid residue corresponding to at least 10% by weight of the total residue and biomass supplied to said pretreatment reactor during said step b′) is in step e) recycled to basic pretreatment step b′).

5. The process as claimed in claim 1, characterized in that at least part of the unconverted solid residue is in step e) recycled to pretreatment step b′), said residue being introduced into the pretreatment reactor.

6. The process as claimed in claim 1, characterized in that the acidic or neutral pretreatment step b) and/or basic pretreatment step b′) includes a prior step a) and/or a′) of impregnation, especially in an impregnation reactor, of the biomass with an acidic, neutral or basic aqueous solution.

7. The process as claimed in claim 6, characterized in that the basic pretreatment step b′) includes a prior step a′) of impregnation, especially in an impregnation reactor, of the biomass with an acidic, neutral or basic aqueous solution, and in that in step e) at least part of the unconverted solid residue is recycled to pretreatment step b′), said residue being introduced into the impregnation reactor during impregnation step a′).

8. The process as claimed in claim 6, characterized in that step a′) of impregnating the biomass and step b′) of basic pretreatment of the impregnated biomass are carried out in respectively impregnation and pretreatment reactors each provided with a biomass feed device, and in that step e) of recycling the unconverted solid residue is carried out by introducing at least part of said residue with the biomass at the beginning of or during treatment into at least one of said feed devices.

9. The process as claimed in claim 1, characterized in that the basic pretreatment step b′) comprises an addition of base to the pretreatment reactor.

10. The process as claimed in claim 1, characterized in that it also comprises:
a step f) of fermenting the hydrolyzed biomass in the form of sugar(s) so as to obtain a fermented biomass comprising at least one alcohol, and in that the solid/liquid separation step d) is carried out on said fermented biomass.

11. The process as claimed in claim 11, characterized in that the enzymatic hydrolysis step c) and fermentation step f) are carried out simultaneously on the pretreated biomass.

12. The process as claimed in claim 10, characterized in that it also comprises:
a step g) of separation or purification, in particular of distillation, of the fermented biomass, and in that the solid/liquid separation step d) is carried out before or after said separation or purification step g).

13. The process as claimed in claim 1, characterized in that it also comprises:
a step h) of combustion of the final unconverted solid residue obtained at the end of the biomass treatment, the heat from which is used in a step of said process that requires heating, especially the heating of a fluid, especially in step b) and/or step b′) of pretreatment by cooking or a step g) of separation by distillation,
and which is optionally preceded by a step i) of drying said residue.

14. The process as claimed in claim 1, characterized in that the solid/liquid separation step d) is carried out by filtration, in particular using a pressing or draining device, such as a filter press or a vacuum filter, a belt filter, a belt press or a centrifuging, decanting or dewatering device or the combination of various devices.

15. The process as claimed in claim 1, characterized in that the lignocellulosic biomass is converted into sugar juice, in particular C5 and C6 juices, after enzymatic hydrolysis, or into alcohol, after fermentation of said sugar juice.

16. A plant for performing the process as claimed in claim 1, characterized in that it comprises:
a) a device, in particular a reactor, for impregnating the biomass with an aqueous solution, in particular an acidic or neutral solution,
b) a reactor for pretreatment by cooking of the impregnated biomass originating from the impregnation device, said cooking optionally being accompanied by steam explosion, to obtain a pretreated biomass, said reactor being provided with means for injecting basic aqueous solution,
c) a reactor for enzymatic hydrolysis of the pretreated biomass to obtain a hydrolyzed biomass,
and in that said plant also comprises
d) a device for solid/liquid separation of the hydrolyzed biomass or of the hydrolyzed biomass subsequently treated in one or more other reactors or devices arranged downstream of the enzymatic hydrolysis reactor c), in order to obtain an unconverted solid residue,
e) means for recycling at least part of said unconverted solid residue obtained in step d) to the impregnation device and/or to the pretreatment reactor when the pretreatment reactor is operated under basic conditions.

17. The process as claimed in claim 1, wherein an amount of unconverted solid residue corresponding to at least 20% by weight of the total residue and biomass supplied to said pretreatment reactor during said step b′) is in step e) recycled to basic pretreatment step b′).

18. The process as claimed in claim 1, wherein an amount of unconverted solid residue corresponding to at least 30% by weight of the total residue and biomass supplied to said pretreatment reactor during said step b′) is in step e) recycled to basic pretreatment step b′).

19. The process as claimed in claim 1, wherein an amount of unconverted solid residue corresponding to at least 40% by weight of the total residue and biomass supplied to said pretreatment reactor during said step b′) is in step e) recycled to basic pretreatment step b′).
